(19) United States
(12) Patent Application Publication
(10) Pub. No.: US 2007/0042507 A1
(43) Pub. Date: Feb. 22, 2007

(54) Composite nanoparticles

(76) Inventors: Shik Chi Tsang, Berkshire (GB); Kin Yip Tam, Cheshire (GB); Xin Gao, Shanghai (CN)

Correspondence Address: MORGAN LEWIS & BOCKIUS LLP, 1111 PENNSYLVANIA AVENUE NW, WASHINGTON, DC, 20004, US

(21) Appl. No.: 10/564,409

(22) PCT Filed: Jul. 16, 2004
(86) PCT No.: PCT/GB04/03103

§ 371 Date: Jul. 7, 2006

(30) Foreign Application Priority Data
Jul. 16, 2003 (GB) 0316675.8
Dec. 29, 2003 (GB) 0330113.2

(51) Int. Cl. G01N 33/551; G01N 33/20

(52) U.S. Cl. 436/524; 436/80; 977/902

(57) ABSTRACT

The method of measuring the partition coefficient of a test molecule comprises incorporating the molecule in a composition of nanoparticles having a porous surface and a first solvent, wherein a second solvent has been absorbed into the porous surface, and said first solvent is immiscible with said second solvent, and then separating the nanoparticles and the first solvent. The amount of the molecule which remains in the first solvent is determined to enable calculation of the partition coefficient. The nanoparticles may have a magnetic core to allow easy separation.

### FIELD OF THE INVENTION

The present invention relates to nanoparticles with a porous surface, methods of making such nanoparticles and their uses in measuring partition coefficients of molecules and in encapsulation of catalytically active species, such as biologically active species. It further relates to a method of depositing a component in pores of a porous material, such as nanoparticles.

### BACKGROUND TO THE INVENTION

The term “nanoparticles” is used to describe particles with dimensions on a nanometre scale. Generally these particles may range in size from around 1 nm up to 1 μm typically having dimensions of between 1 nm and a few hundreds of nanometres. Due to their small size, nanoparticles have a very large surface area to volume ratio. This feature explains the reason why many of the uses of nanoparticles are in processes requiring a maximised surface area with the lowest possible volume such as many heterogeneous catalysis reactions.

Nanoparticles can vary in their internal structure. The simplest particles consist of just a single material whilst more complex particles may have a core region with one or more different layers, formed from different materials, arranged around it.

There are a number of methods of making nanoparticles ranging from simple grinding and milling techniques, through deposition from a microemulsion and polymerisation of emulsions to electric arc vaporisation of a material. The method used depends upon the complexity of the particle which is required e.g. the number of layers of different material in the particle, how the different layers interact and other well defined parameters.

Whilst nanoparticles made from a single material are the simplest to manufacture, by simple milling techniques, particles with an outer coating on them are nevertheless widely used in order to protect the inner core of the particle from chemical or physical degradation.

Methods of making nanoparticles with a variety of different core materials and a surface layer are well known. U.S. Pat. No. 6,548,264 discloses a range of particles with a silica coating on the outside and methods of making them using microemulsions.

International application PCT/GB2003/000029 (Reading University), published as WO 03/057359, also describes a method of making silica particles having a magnetic core. Methods of making nanoparticles of other coated materials, such as alumina, titania or zirconia by sol-gel technology or related techniques are known.

### SUMMARY OF THE INVENTION

The present invention makes use of the porosity of the surface layer of nanoparticles, in the application of such particles to new uses. The porosity can be controlled in the manufacture of such particles. The manufacturing method also permits the introduction of molecules of interest in the core of the particle, inside the porous coating or layer, the porosity of the particle providing access to the entrapped molecule.

In one aspect of the present invention nanoparticles are prepared and used in a process for determination of the partition coefficient of a molecule in a solvent system consisting of two immiscible solvents. The partition coefficient of a molecule is dependent upon the solvent system in which it is measured and gives a numerical assessment of how the molecule is distributed between the two solvents at equilibrium. This has one particular use in pharmaceutical drug development.

The present state of the art for measuring partition coefficient values is described in “Pharmacokinetic Optimization in Drug Research: Biological, Physicochemical and Computational Strategies” (B. Testa, H. van de Waterbeemd, G. Folkers, R. Guy (editors), Verlag Helvetica Chimica Acta, Zurich, 2001). In the most generally applied measurement, a test molecule is dissolved to a known concentration in a first solvent e.g. water. A known amount of this solution is then added to a known amount of a second solvent e.g. n-octanol and the two phases are well mixed. The system is then allowed to reach concentration equilibrium. Finally the phase containing the first solvent is separated out and the concentration of the test molecule in this solution is measured. From this value, a partition coefficient (P) can be determined.

A number of problems arise with this method of determining the partition coefficient. When the two immiscible phases are mixed, it is necessary to allow them to equilibrate for a long time to reach concentration equilibrium. This is due to the relatively low contact area between the two phases. Also, the separation of the two phases requires a visual assessment of the position of the inter-phase boundary. If a molecule is highly soluble in one phase, it may be necessary to use a very small amount of that solvent which makes assessment of the position of the inter-phase boundary difficult. In addition, the fact that the inter-phase boundary must be visible for the phases to be separated requires relatively large volumes of solvents to be used. This generates large volumes of hazardous waste and adds to the cost of the procedure.

The combination of these problems makes this process a costly and time-consuming one, especially when performed on an industrial scale.

The present invention in one aspect seeks to overcome the above problems by using nanoparticles in a method of measuring the partition coefficient of a test molecule.

In a first aspect therefore the invention provides a method of determining the partition coefficient of a chemical compound between two solvents in a mixture containing a first solvent and a body of nanoparticles, wherein a second solvent is absorbed in the pores of nanoparticles. A body of nanoparticles having the solvent absorbed in them can provide a predetermined quantity of the solvent, which can be very small, allowing determination of extreme partition coefficients. The nanoparticles can be easily separated from the first solvent, for example when the nanoparticles have a magnetic core permitting magnetic separation. Other methods of separation are available such as centrifugation, or changing the dielectric constant of the system, e.g. by addition of another solvent, to cause precipitation of the nanoparticles; the nanoparticles employed in this aspect of the invention therefore may consist only of the porous material.

It has been shown that the use of nanoparticles in the measuring of partition coefficients does not affect the results i.e. the nanoparticles do not significantly influence the value of the partition coefficient obtained. The procedure can be quick, since the equilibrium distribution of the measured compound is obtained rapidly, and economical since the easy and-effective separation of the nanoparticles allows small quantities of one or both solvents to be used.

It is desirable, for accurate determination of the partition coefficient, that the first solvent phase is pre-saturated with the second solvent phase and vice versa. Therefore it is not necessary that the two solvents are mutually wholly insoluble; what is important is that they form two immiscible phases, and the term “immiscible” is used here in this sense.

The present invention also consists in compositions containing nanoparticles which are useful in this method of determining partition coefficients. There is provided a mixture of nanoparticles having a porous outer coating, and a first solvent, wherein a second solvent is absorbed into the porous coating of the nanoparticles and wherein said first and second solvents are immiscible. Such a composition is stable e.g. as a colloidal dispersion of the particles in the first solvent, has a long shelf life and permits easy and accurate dispensing of a predetermined quantity of the second solvent. In this composition, preferably the second solvent phase is wholly absorbed in the nanoparticles, i.e. does not appear freely outside the nanoparticles.

The amount of the nanoparticles containing the second solvent per unit volume of first solvent (i.e. also per unit volume of the total composition) can thus be predetermined, i.e. known, and fixed for a particular composition. The nanoparticles can be uniformly distributed in the first solvent, as a colloidal solution. Thus the ratio of the volumes of the two solvents is accurately predetermined. Volumetric dispensing of a quantity of the composition can therefore be performed, providing in an easy manner any desired volume of the two solvents in a predetermined ratio. High accuracy can be achieved.

The composition is preferably stored in a sealed container, to prevent evaporation.

The first solvent in which the nanoparticles are suspended is preferably aqueous e.g. water or an aqueous solution or a water-containing phase.

The second solvent which is absorbed into the porous outer coating of the nanoparticles may for example be a water-immiscible solvent, e.g. n-octanol, cyclohexane, alkane (C₆-C₁₀), chloroform, propylene glycol dipelargonate (PGDP), 1,2-dichloroethane, olive oil, benzene, toluene, nitrobenzene, chlorobenzene, tetrachloromethane, oleyl alcohol, 4-methylpentan-2-ol, pentan-1-ol, pentan-2-ol, isobutanol, butan-1-ol, 2-methylbutan-2-ol, butan-2-ol, butan-2-one, diethyl ether, isoamyl acetate, ethyl acetate, etc.

Both solvents are preferably free of any biologically active compound, particularly any pharmaceutically active compound.

A method of forming this composition of the invention, using supercritical fluid to form the particles containing solvent, is described below.

An alternative form of composition provided by the invention, also suitable for accurate dispensing of a predetermined amount of a solvent (i.e. the solvent called the second solvent above) in a form convenient for a quantitative analytical procedure such as the partition coefficient determination herein described, is a composition comprising nanoparticles each having a porous surface and the solvent adsorbed in the pores of the nanoparticles in a predetermined amount per unit weight of the composition. In this composition, preferably there is no free solvent (i.e. it is all absorbed in the nanoparticles), so that the composition is effectively a particulate solid and is dispensable by weighing (gravimetrically). The amount of the solvent is thus predetermined for unit weight of the composition. This composition also is preferably stored in a sealed container, to prevent evaporation of the solvent. The solvent may be substantially free of any solute, e.g. free of any biologically active compound. This composition can be accurately mixed with a desired quantity of another solvent (called first solvent above), to obtain a composition of two solvents as described above; this may be done for example by user, immediately prior to use. A method of forming this composition of the invention, using supercritical fluid, is described below.

The invention in a second aspect arises from the finding that a catalytically active species, especially a biologically active species, especially a biological catalyst, can be entrapped in the cores of porous nanoparticles in a state in which its catalytic activity is maintained and in which substrate molecules can access it via the pores of the particle for catalytic reaction to occur. This is due to the porous coating having a pore size smaller than the size of the biologically active species. One advantage is that the bioactive species may be entrapped without chemical bonding, so that it is essentially in its free state of optimum nature. Its activity may therefore not be impaired or altered, in contrast with known techniques in which molecules are chemically bonded to a support.

By control of particle size, and in particular core size, it is possible to provide a body of nanoparticles having a known, reproducible quantity of the entrapped species. The core size may be such that only one molecule of the bioactive species is present; in this case, in a population of the nanoparticles, some may contain no catalytic molecule and some may contain more than one. It is possible therefore to provide a population or assembly of nanoparticles containing on average not more than one molecule of the catalytically active species per particle.

One advantage of this entrapment is to reduce aggregation or agglomerization of the bioactive species (reduce the formation of dimer, trimer, tetramer and so on) by means of the coating, which reduces the extent of deactivation.

The nanoparticles containing catalytically active species in this aspect of the invention can be employed in many applications, e.g. enzymatic reactions and other catalytic reactions, assay methods (e.g. by binding of target molecules to the entrapped species such as antigen-antibody reactions; protein-drug binding; bioreceptor-antigen binding, oligonucleotide recognition; biotin-streptavidin reactions), and as biosensors etc. An important advantage is to trap a free form of bulky bioactive species inside the core of the nanoparticle with a porous coating of tailored size. This prevents leaching of the trapped species to solution through the coating. On the-other hand the pore size of the coating allows the exchange of small molecules (smaller than the pore size), permitting access to the trapped molecules freely. Separation can be therefore achieved using trapped core magnet(s) or by other means. As a result, the porous coating of the composite nanoparticles can be regarded as a ‘nano-membrane’ for molecular recognition and separation.

In addition, the nanoparticles of the present invention encapsulating catalytically active species can allow catalysis to be performed on a small scale and allowing simple separation of products from a heterogeneous catalyst.

As described above, the present invention provides a composition of nanoparticles having porous coatings which are in a first solvent and carry a second solvent adsorbed in their porous coating. The invention further provides a composition of nanoparticles having a solvent adsorbed in their pores in a predetermined amount per unit weight of the composition.

The present inventors have found a novel way to deposit a material, such as a solvent liquid, in the pores of nanoparticles with a high degree of quantitative accuracy. This method is applicable to the deposit of a material in any porous surface, such as a surface of a large body, or the surface of particles of any size, as well as nanoparticles.

According to the invention in yet another aspect, therefore, there is provided a method of depositing or dispersing a component in pores of a porous material, by contacting the porous material with a solution of the component in a supercritical fluid. Suitably the supercritical fluid is removed by depressurising it and allowing it to evaporate.

A suitable supercritical fluid is carbon dioxide (SC—CO₂) which becomes supercritical at easily manageable temperatures and pressures. Examples of other substances which can form suitable supercritical fluids are ethane, water, butane, ammonia and noble gases such as Ar, Xe and Kr.

Components which are soluble in SC—CO₂ are for example organic molecules such as hydrocarbons (both aliphatic and aromatic), halocarbons, aldehydes, esters, ketones and alcohols, e.g. aliphatic alcohols of 1 to 12 carbon atoms, such as n-octanol. Particularly, in one aspect the invention may be applied to relative low molecular weight (e.g. ≦200) solvent compounds, but the invention also includes the deposition or dispersion of other molecules such as macromolecules such as bio-species and drug molecules, having for example mol. wt.≦500, particularly 200-500. Two or more components may be deposited or dispersed simultaneously.

The invention further provides a method of preparing a composition containing two components comprising preparing porous particles containing a first component in a predetermined amount by a method using supercritical fluid as described above, and adding the particles containing the first component to a liquid second component. The two components are typically immiscible. The second component may for example be aqueous. Accurate ratios of the two components may be achieved, even when the second component is in large excess, e.g. the ratio by volume is 100:1 or greater, e.g. in the range 100:1 to 3000:1, preferably 500:1 to 1500:1.

### BRIEF DESCRIPTION OF THE FIGURES

FIG. 1 shows the correlation between log D results achieved by measurement using nanoparticles and literature values.

FIG. 2 shows the correlation between log D results achieved by measurement using nanoparticles and values obtained using the prior art method.

FIG. 3 shows the magnetic field response of the particles obtained using the method of example 1.

FIG. 4 shows a transmission electron microscopy (TEM) micrograph of the silica coated particles produced by the method of example 1.

FIG. 5 shows an x-ray diffraction (XRD) pattern of the silica-coated Fe₃O₄ nanoparticles obtained in example 1 recorded using a wavelength of 1.54056 nm.

FIG. 6 shows a thermogravimetric (TG) analysis of the silica coated Fe₃O₄ nanoparticles obtained in example 1.

FIG. 7 shows a thermogravimetric (TG) analysis of the silica coated Fe₃O₄ nanoparticles in which the silica coating has been saturated with n-octanol.

FIG. 8 shows two infra red (IR) spectra: a) is of the silica coated Fe₃O₄ nanoparticles; b) is of the silica coated Fe₃O₄ nanoparticles which have been treated with chlorotrimethyl silane (CTMS).

FIG. 9 shows an XRD pattern of the Fe₂CoO₄ nanoparticles obtained in example 5.

FIG. 10 shows a UV-visible spectrum of a penicillin V solution in the presence of β-lactamase I.

FIG. 11 shows a UV-visible spectrum of a penicillin V solution in the presence of a micellar solution of β-lactamase I.

FIG. 12 shows a UV-visible spectrum of a penicillin V solution in the presence of β-lactamase I which is encapsulated inside a porous silica coating.

FIG. 13 is a pressure-temperature diagram of carbon dioxide.

FIG. 14 is a schematic view of apparatus for deposition onto particles using supercritical CO₂.

FIG. 15 is a graph of absorbed n-octanol against amount of octanol added.

FIGS. 16 to 18 are correlation curves for the results given in Table 3.

### DETAILED DESCRIPTION OF THE EMBODIMENTS

Formation and Properties of Porous-Coated Nanoparticles.

As mentioned above solid nanoparticles having a core surrounded by a porous coating can be made by a method which includes the steps of:

(a) forming, in a liquid medium, colloidal particles containing a core species and colloidally stabilized by organic stabiliser(s) or stabilized as micellar aggregates (e.g. stabilised water droplets embraced by surfactant molecules), and

(b) forming a porous coating around the colloidal particles by hydrolyzing a precursor compound in the region of the interface between the colloidal particle or micellar particle and the liquid medium.

Preferably the nanoparticles are aged, e.g. for an hour to weeks, preferably 2-5 days, before removal from the colloidal system, in order to establish the porous coating to the desired thickness.

The porous coating formed around the core of the particle may be formed from a range of porous materials such as alumina, silica, titania, zirconia or carbon. Preferably the porous coating is formed from silica by hydrolysis of a silicon-containing compound at the interface region of the colloidal suspension.

The compound which is hydrolyzed may be an alkoxy silane compound, i.e. a compound containing at least one Si—OR linkage, where R is alkyl of preferably 1-8 carbon atoms, more preferably 1-4 carbon atoms, such as tetraethyl ortho silane (TEOS, Si(OC₂Hs)₄); and chloro-, bromo-, hydro- and metallo-silanes, (containing Si—Cl, Si—Br, Si—H or Si—M bonds where hydrolysis occurs). Alternatively, the compound which is hydrolysed may be an analogous alkoxy, halo, hydro compound of titanium, aluminium or zirconium (or an intermetallic compound) such as titanium isopropoxide or titanium tetrachloride.

After hydrolysis of the above compound(s) the described aging process allows cross condensation of the —OH species, forming a three-dimensional gel (with e.g. Si—O—Si or Ti—O—Ti linkage) embracing the particle therein.

For the formation of a carbon coating, the colloidal particles formed in step (a) are separated from the colloidal suspension and are pyrolysed so that the organic surfactant coating around the particles decomposes to form a porous carbon outer coating around the nanoparticle core. Other carbon precursor(s) such as polyvinyl alcohol, phenol/polyphenols, polysaccharides, etc could be used for the porous carbon formation.

In a preferred form of the method in step (a) the colloidal particles are made by forming an emulsion having dispersed phase droplets or micelles stabilized by the surfactant and containing a dissolved compound of a core material and causing the core species to precipitate thereby forming the colloidal particle inside the micelles. The precipitation may be caused by addition of alkali or ammonia.

Preferred surfactants used for stabilising the colloidal particles include cetyltrimethylammonium bromide (CTAB), oleic acid, polyvinylpyrrolidone (PVP), non-ionic surfactants such as AOT, TX100, etc.

The porous material may have at its surface functional groups, e.g. OH groups, for the chemical (e.g. covalent) attachment of other species, such as biochemical or biological species (e.g. peptides, markers, cognate binding partner, solubilizers) or attachment of the nanoparticle to a substrate with or without the use of linker molecules. Immobilisation of charged species on charged surface at defined pH by electrostatic interactions is also included.

A plurality of metal-containing species of different metals may be included in the colloidal particles, and thus in the core of the nanoparticles produced. Typically such a metal-containing species is selected from metal, alloy, metal oxide, metal hydroxide and metal carbide. Preferably the metal-containing species is ferromagnetic (enabling magnetic separation of the nanoparticles) or super-paramagnetic, or single domain magnetic nanoparticles are employed. Magnetic materials which may be included in the core of the nanoparticles include magnetite (Fe₃O₄), maghemite (γFe₃O₄), greigite (Fe₃S₄) and Fe₂CoO₄.

The cores of the nanoparticles may alternatively or additionally comprise a catalytically or biologically active species. Preferred biologically active species include enzymes and proteins. Examples are lactase, metallothionin, cytochrome (such as cytochrome b, cytochrome c or cytochrome P450), blood albumin(s), carboxylesterases, kinase, short nucleic acid oligomers, antibody species and enzyme indicators in blood/liver tissues. referred catalytic species include inorganic catalyst compounds (e.g. formed by “ship in a bottle chemistry”) such as heteropolyacids, metallothioleins, corands, coraplexes, spherands, spheraplexes, cavitands, host-guest catalysts, and intercalated catalysts, etc.

Where the cores of the nanoparticles include a catalytically or biologically active species, it is preferred that the porous coating has a pore size smaller than the catalytically or biologically active species so that the active species is retained inside the coating of the nanoparticle.

Furthermore, it is preferred that the porous coating of the nanoparticle has a pore size which is large enough to allow small molecules to pass through. In particular, it is preferred that where a catalytically active species is encapsulated in the core of the nanoparticle, the pore size of the porous outer coating is larger than the size of both the reactant and the product of the catalytic reaction. In this case, a reactant molecule may pass through the porous coating of the nanoparticle, interact with the catalytically or biologically active species retained inside the nanoparticle and products from the interaction may pass out through the porous coating.

The nanoparticles preferably have an average diameter in the range 1 nm to 1 μm, more preferably 1 to 100 nm. The porous coating may have any desired thickness, but preferably has an average thickness in the range 1 to 100 nm, preferably 1 to 50 nm. Where the core of the nanoparticle comprises a catalytically or biologically active species, the diameter of the core may be between 1 and 10 nm and is preferably between 1 and 5 nm.

Use of Porous-Coated Nanoparticles in the Measurement of the Partition Coefficient of a Test Molecule.

The present invention provides a method of attaining partition of a test molecule between two immiscible solvents through the use of porous nanoparticles. The method comprises the step of mixing the test molecule with a first solvent of a colloidal suspension comprising nanoparticles with a porous outer coating wherein a second solvent is absorbed into the porous outer coating, the nanoparticles being suspended in the first solvent which is immiscible with the second solvent. The test molecule dissolves partially in the second solvent, and is retained in the porous outer coating of the nanoparticles, and partially in the first solvent.

The step of obtaining the composition containing the compound being tested (the analyte), the nanoparticles, the first solvent containing the nanoparticles and the second solvent in the pores of the nanoparticles, may be carried out in any suitable way. For example the analyte may be introduced as a solution, e.g. in a buffer, which is mixed into the composition of the nanoparticles, and the first and second solvents. The analyte solution is miscible with the first solvent, both being for example aqueous. Alternatively a known amount of the analyte dissolved in a small amount of a solvent, such as DMSO, is injected into the composition of nanoparticles and first and second solvents. Another alternative is to introduce the nanoparticles containing the second solvent into a solution of the analyte in the first solvent.

In order to optimise the speed with which partition of a test molecule is achieved, it is desirable to maximise the contact area between the two immiscible solvents. This can be achieved by forming particles with a high ratio of surface area to volume, i.e. the smaller the diameter of the particle, the faster partition will be achieved. In addition, the porous coating on the nanoparticles should absorb as much solvent as possible in order to speed up the partitioning of the test molecule. As such, small particles with large pore volumes are preferred. If the features of the porous coating which favour a fast partitioning of the test molecule are optimised, partitioning times in the region of 1-10 minutes or less may be achieved using nanoparticles of the present invention. This represents a significant improvement over the partitioning times achieved in the prior art.

In this method the compositions comprising solvents and nanoparticles described above may be employed.

The present method of attaining partition of a test molecule between two immiscible solvents may be used to determine the value of the partition coefficient for the test molecule.

The following terms will be used in the discussion of partition coefficients:

log P is the standard value quoted for the partition coefficient of a test molecule where P=[C]_{organic}/[C]_{aqueous}. Unless otherwise specified, these values are recorded for an octanol-water biphasic system using the molecule in its electronically neutral form.

log D is the most commonly used value in this specification. This refers to the partition coefficient of a test molecule at a specified pH value. In calculating these values the following relation is used for D:
D_{pH}=f_{N}×P_{N}+f_{I}×P_{I}

where f_{N} and f_{I} are the molar fractions of the neutral and ionised forms of the test molecule respectively and P_{N} and P_{I} are the P values for the neutral and ionised forms of the test molecule respectively.

One method of measuring the partition coefficient (either log P or log D) value for a test molecule comprises the steps of:

a) providing a composition of nanoparticles, with a porous surface and a first solvent wherein a second solvent has been absorbed into the porous surface, and said first solvent is immiscible with said second solvent;

b) incorporating a molecule to be tested in a composition of step a); and

c) separating the product of step b) into two components, the first comprising the nanoparticles and the second comprising the first solvent; and

d) the amount of the molecule to be tested which remains in the first solvent may be determined to enable calculation of the partition coefficient.

Step c) of this method may be achieved by e.g. filtration or centrifugation of the product of step b) to separate the mixture into the two components comprising the nanoparticles and the supernatant solution, or by other separation methods mentioned above. In the case where the core of the nanoparticle contains a magnetic material, a magnetic field may alternatively be used to perform step c) of the above method. In this case, a magnetic field applied to the product of step b) can be made to precipitate the nanoparticles from the reaction mixture.

Step d) of the method of measuring the partition coefficient may be achieved by any analytical technique through which the concentration of the test molecule in a solution can be determined. These techniques may include nuclear magnetic resonance (NMR), titration, UV-visible spectroscopy, fluorescence, phosphorescence, high-performance liquid chromatography (HPLC), gas chromatography (GC), mass spectroscopy (MS), GC-MS, gravimetric, surface plasma and electro-analytic techniques. Preferably the technique used in step d) does not require further processing of the supernatant solution and may be performed without removal of a sample from the reaction vessel. In a most preferred embodiment, the technique used in step d) is UV-visible spectroscopy.

In the preferred case where UV-visible spectroscopy is used to determine the concentration of the test molecule in the supernatant solution, the following equation may be used to calculate log D:
log D=log {[(A1−A2)/A2]×V₁/V₂}
where A1=UV-visible absorption of the test molecule in the supernatant phase before partitioning.

A2=UV-visible absorption of the test molecule in the supernatant phase after partitioning.

V₁=Volume of first solvent (with which the nanoparticles are mixed).

V₂=Volume of second solvent (absorbed into the porous outer coating of the nanoparticles).

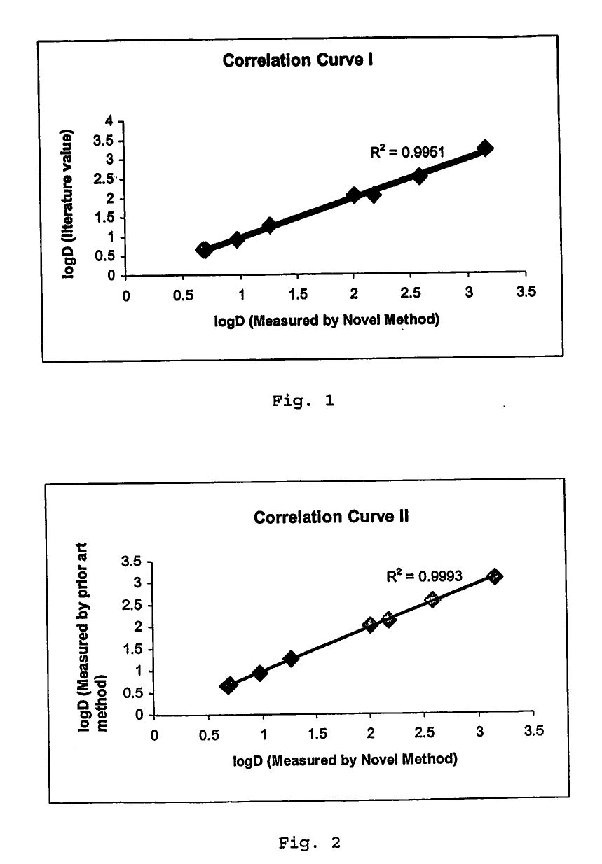

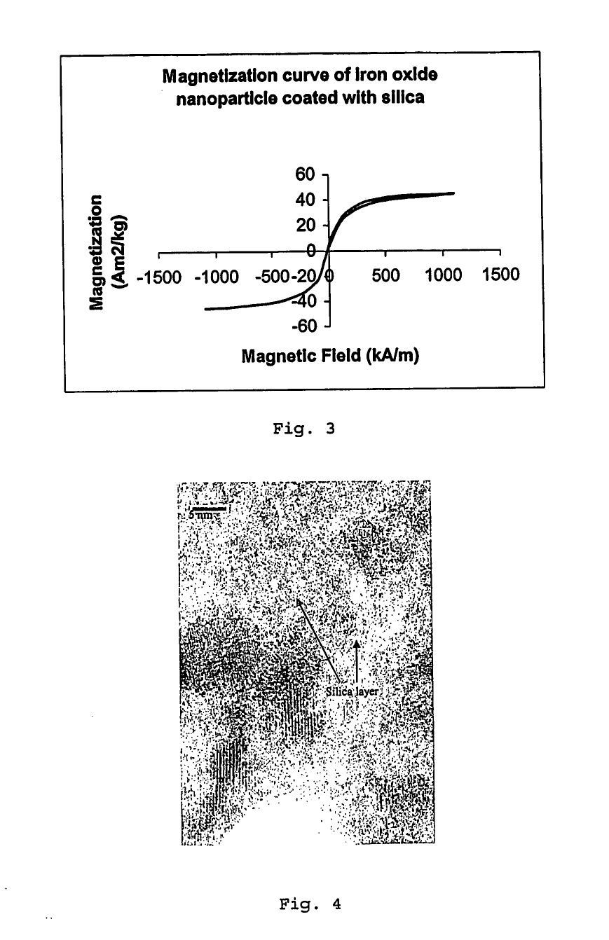

Correlation of the log D values measured by the method of the present invention and those measured by the prior art method is demonstrated by FIGS. 1 and 2.

The selection of the volume ratio of second solvent (absorbed into the porous outer coating of the nanoparticle) to the first solvent is made depending upon the approximate solubility of the test molecule in the two solvents (which if not known previously may be estimated or may be arrived at by experiment). In the present invention, the ratio of first solvent to second solvent may be between 3000:1 and 1:1. Typically the ratio of first solvent to second solvent is greater than 50:1 and preferably 100:1 or greater, and may be as high as 500:1 or greater, e.g. in the range 500:1 to 1500:1.

This method of measuring the partition coefficient of a test molecule has a number of distinct advantages over the prior art methods.

First, the partition of the test molecule is achieved faster than in the prior art for the reasons already discussed.

Secondly, in the case where the nanoparticle core contains a magnetic material, the mixture used to measure the partition coefficient can be easily separated into a nanoparticle component and a supernatant solution by application of a magnetic field to the solution. Typically separation of these two components of the mixture can be achieved in a matter of seconds.

Thirdly, as one of the solvents is absorbed into the porous coating of the nanoparticles, problems with evaporation of volatile solvents during the measurements may be overcome. In the prior art method, great care must be taken when measuring the partitioning of a compound into a volatile solvent to avoid that the solvent evaporates, changing the volume of the solvent, during the measurement making calculation of the partition coefficient complex. In the present method however, the volatile solvent may be absorbed into the porous outer coating of the nanoparticles which lowers the rate of evaporation of the solvent. Coupled with much faster partitioning of the test molecule, leading to overall lowering of the measurement time, this allows partitioning of a test molecule into a volatile solvent using the method of the present invention.

Fourthly, the present method does not rely on a visual determination of the solvent interface in order to achieve separation of the two solvents since separation is achieved by separation of the nanoparticles e.g. by magnetic separation, filtration or centrifugation. Hence this method of measuring the partition coefficient of a test molecule can be performed using much lower volumes of solvent than the prior art method. A lower volume of solvent lowers the cost of the measurement and facilitates automation of the process. Additionally, measurements using a lower volume of solvent produce less hazardous waste which further lowers both cost and environmental impact of the process.

Finally, the present process can be used to measure partition coefficients even in the case where a test molecule is highly soluble in one of the solvents. As mentioned above, the suitable ratio of first solvent to second solvent is determined by the solubility of the test molecule in each solvent. In the method of the present invention, a wider range of first solvent to second solvent ratios may be used in the measurement of partition coefficient values. This is due to the fact that separation of the solvents prior to measurement of the concentration of the test molecule does not require a visual determination of the boundary between the first and second solvents. As such ratios of between 3000:1 to 1:1 first solvent:second solvent can be used. The use of small quantities of solvents, test molecule and composite nanoparticles coupled with the fast spectroscopic determination of test molecule concentration in supernatant allow a rapid evaluation of partition coefficient. Thus, a high throughput screening of a wide variety of test molecules in robot-friendly manner can be established.

Catalytic Species Encapsulated within a Nanoparticle with a Porous Outer Coating.

The advantages of nanoparticles within a porous outer coating encapsulating catalytically active species are wide ranging due to the variety of outer coatings and catalytically active species which can be envisaged. The use of the porous-coated nanoparticles of the present invention in heterogeneous inorganic catalysis is envisaged, as is also their use in containing biologically active species.

In one aspect nanoparticles with a core containing a magnetic material are particularly useful as their catalytic activity can be exploited in suspension and separation of the catalyst from the product of the reaction is easily achieved using a magnetic field.

A further advantage of the nanoparticles of the present invention when the core material comprises a biologically active species, is that the biologically active species may not be chemically altered compared with its free state, for example by attachment of solubilising groups or linker groups to bind the species to a substrate. This means that the physical structure of the contained species is not altered by binding to pendant groups or the like. Thus in this aspect of the invention the species behaves in a similar way to the non-encapsulated form.

It is also known that a suspension of biologically active molecules, such as enzyme molecules, aggregates in solution if the concentration of the molecules is raised above a certain threshold. This limits the use of biologically active species at high concentration in suspension, since aggregation will lead to a drop in the surface area to volume ratio of the molecule and hence a potential lowering in the number of binding sites available to interacting molecules. In the present invention however, the porous outer coating surrounding the biologically active species prevents aggregation of the molecules and allows the species to be present in suspension to higher concentration than with the prior art methods.

The broad applicability of the encapsulation method to various core materials and range of potential porous outer coatings results in a huge variety of potential applications for the nanoparticles of the present invention. Applications which are envisaged include assay methods for a variety of drug molecules, measurement of molecular binding constants, catalysis reactions (in both biological and chemical systems), biosensor applications, antibody-antigen, storage and release, etc. One example is encapsulation of albumin, for study of drug/albumin interaction and/or determination of binding constant.

### EXAMPLES

### Example 1

### Formation of Porous-Silica Coated Fe₃O₄ Nanoparticles

Formation of a microemulsion was carried out using de-ionized water, excess pre-dried toluene and ionic surfactant (CTAB). Typically, the experiment was carried out at room temperature. The microemulsion was formed as follows: 0.02 mol CTAB (99%, Aldrich) was added into bo0g dried toluene (99+%, Fisher) under vigorous stirring to create a well-distributed suspension of CTAB in toluene. 0.3428 g FeCl₂.4H₂O and 0.9321 g FeCl₃.6H₂O (both 99%, Aldrich) were dissolved in 6.2 g water. This solution was added slowly in droplets into the toluene suspension of CTAB in nitrogen atmosphere. After stirring for 4 h, NH₃ solution (18.1 M, 1 ml, Fisher) was then added to the mixture. After an hour the whole system turned black in color. It has been previously shown that the addition of Fe²⁺, Fe³⁺ and ammonia will produce magnetic Fe₃O₄ precipitate. At this point tetraethyl orthosilicate (TEOS) (99%, Aldrich) was added into the reaction mixture. Nitrogen was bubbled through the mixture for one hour. The ammonia solution (high pH) catalyzed hydrolysis/condensation of the TEOS into silica-gel. The silica over-layers were aged for 5 days in suspension. Finally, the precipitate was isolated by magnetic separation means and washed several times with hot ethanol, water and acetone to remove surfactant and organic solvents. The precipitate was then dried at room temperature resulting in a deep brown powder.

### Example 2

### Analysis of the Product of Example 1

The product obtained in example 1 was analysed using a variety of techniques.

The particles showed a strong magnetic response upon exposure to magnetic field showing a super-paramagnetic response(see figure number 3)

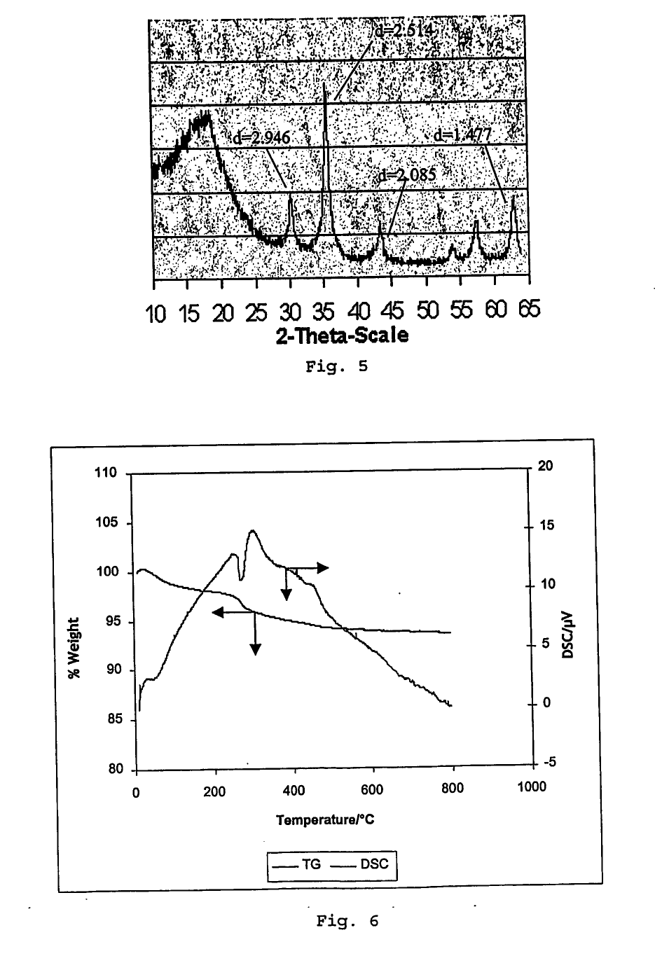

The Fe₃O₄ nanoparticles are shown by transmission electron microscopy (TEM) to be approximately 12 nm in diameter (see FIG. 4 whereas calculations from X-ray diffraction (XRD) measurements indicate that the particles are around 17 nm in diameter (see FIG. 5).

The chemical composition of the nanoparticles was measured by energy dispersive spectrometry (EDS) (see table 1)

TABLE 1
Energy Dispersive Spectrometry Analysis of the
synthesized nanoparticles.

| | Element Atomic (%) | | |
|---|---|---|---|
| | Fe | O | Si |
| Site 1 | 24.21 | 60.28 | 15.51 |
| Site 2 | 24.60 | 63.94 | 11.46 |
| Site 3 | 23.87 | 62.46 | 13.67 |
| Site 4 | 23.59 | 60.94 | 15.47 |
| Site 5 | 24.84 | 61.50 | 13.66 |
| Site 6 | 23.39 | 62.59 | 14.02 |
| Average | 24.08 | 61.95 | 13.97 |
| Value | | | |

Calculations from table 1 suggest that the composition of the silica-coated Fe₃O₄ individual particles is Fe₃O_{4.24}.1.74SiO₂. Further experiments forming particles using the method of example 1 but varying the TEOS concentration suggest that nanoparticles with a composition of Fe₃O_{4.1}.0.21SiO₂ can also be obtained. This suggests that tailoring of the thickness of the silica coating on the nanoparticles is possible using the method of example 1.

### Example 3

### Measurement of the Porosity of the Silica Coating on the Nanoparticles Produced in Example 1

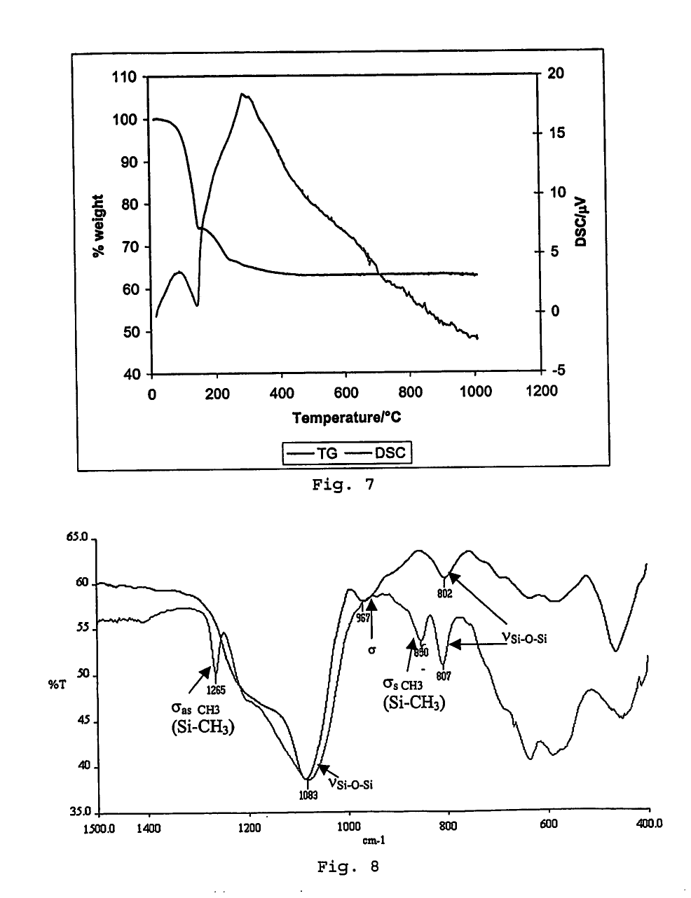

An estimation of the maximum amount of n-octanol which can be trapped in the pores of the silica coating of Fe₃O₄ nanoparticles produced by the method of example 1 can be obtained by thermogravimetric (TG) analysis (see FIGS. 6 and 7). The values obtained from TG analysis of the product prepared by the method of example 1 suggests that the silica coating can absorb up to 0.54 ml per 1 g nanoparticles. BET (>300 m² per gm of silica) and pore size measurements (pore size range from 0.5-3 nm) also indicate that the composite nanoparticles are porous in nature.

### Example 4

### Capping of Surface Hydroxyl Groups

Porous-silica coated Fe₃O₄ nanoparticles obtained by the method of example 1 were further modified to cap surface hydroxyl groups on the silica coating with trimethyl silyl (—Si(CH₃)₃) groups. Excess CTMS was allowed to flow through a fixed bed of dried silica-gel coated Fe₃O₄ in nitrogen gas at 120° C. IR spectra of the porous-silica coated Fe₃O₄ nanoparticles before and after treatment with CTMS are shown in FIG. 8 showing the decrease in intensity of the Si—OH signal (˜967 cm⁻¹) and appearance of the Si—CH₃ signal (˜850 cm⁻¹ and ˜1265 cm⁻¹) which indicates the capping of the —OH groups on the silica surface.

### Example 5

### Formation of Porous-Silica Coated Fe₃O₄

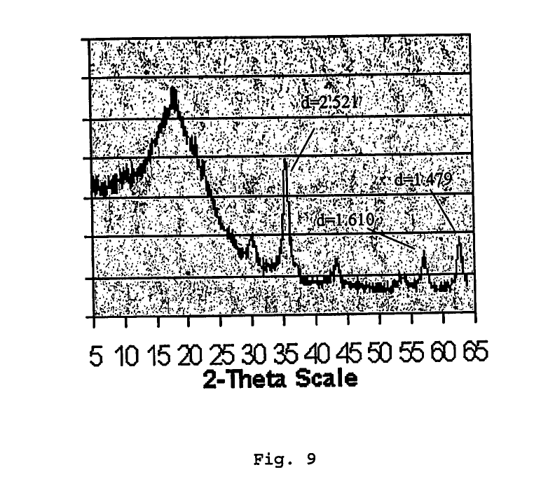

Fe₂CoO₄ nanoparticles were produced and coated with a porous silica coating by the same method as in example 1. In this case, equal molar amounts of FeCl₃.6H₂O (the same amount used-as described in Example 1) and CoCl₂.xH₂O were dissolved in water which was added to the toluene suspension of CTAB in the same manner as example 1. The size of the Fe₂CoO₄ particles was measured by XRD (FIG. 9) as approximately the same size as the Fe₃O₄ particles formed in example 1.

### Example 6

### Measurement of Log D Values Using Porous-Silica Coated Nanoparticles.

Potassium dihydrogen orthophosphate (99%, Aldrich) 0.1 mM aqueous solution, pH value adjusted to be 7.4, was used as a buffer solution in the following measurements. The test molecule was dissolved into the buffer solution that had already been pre-saturated with n-octanol in a glass vial. The test molecule concentration was kept at about 1×10⁻⁵ M. n-octanol, 10 to 100 μl, pre-saturated with the buffer solution was physically absorbed onto the porous-silica coated Fe₃O₄ nanoparticles (obtained by the method of example 1) by capillary action. All the octanol added was completely adsorbed, so that the nanoparticles containing it appeared as a dry powder, and no oily droplets could be seen. The nanoparticles, containing a known amount of n-octanol, were allowed to disperse into a known concentration of the test molecule solution. The volume ratio of aqueous solution to n-octanol in the mixture was set at 100:1. The glass vial was sealed and put in an orbital shaker. The shaking speed was carefully controlled to avoid any n-octanol droplets detaching from the composites (visually). After shaking an external magnet was placed near the bottom of the vial. Magnetic induced precipitation was achieved in a few minutes or less. UV-visible absorptions of the test molecule analyte in the supernatant aqueous phase before and after precipitation were measured with baseline correction. The log D value was then obtained using the equation below:
log D=log {[(A1−A2)/A2]×V_{w}/Vₒ}
where: A1=UV-visible absorption of the test molecule in the supernatant phase before partitioning.

A2=UV-visible absorption of the test molecule in the supernatant phase after partitioning.

Vₒ=Volume of n-octanol(absorbed into the porous outer coating of the nanoparticles).

V_{w}=Volume of water (with which the nanoparticles are mixed).

Unless otherwise stated the volume ratio of the first solvent (water) to the second solvent (n-octanol) was fixed to be 100:1.

Partition coefficient values of some test molecule analytes were measured with the surface —OH groups of the nanoparticles capped with trimethyl silyl (TMS) groups to compare with un-capped nanoparticles.

For comparative purposes the partition coefficient of each test molecule analyte was independently measured by a prior art “shake-flask” method with the same test molecule concentration and the phase volume ratio. The results of these measurements are shown in table 2 below.

TABLE 2

| | | | LogD value | Mean logD | LogD value | Mean logD |
|---|---|---|---|---|---|---|
| | | | measured | value measured | measured by | value |
| | | LogD value | (Prior art | (Prior art | present | measured by |
| | | from | method) | method) | invention | present invention |
| Molecule | Structure | literature | (pH = 7.4) | (ph = 7.4) | (pH = 7.4) | (pH = 7.4) |
| Benzamide | 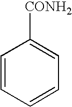 | 0.660 | 0.643 0.651 0.663 0.654 0.658 | 0.654 ± 0.0094 | 0.0639 0.656 0.704 0.719 0.673 | 0.678 ± 0.0460 |
| 4-Nitroansiole | 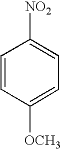 | 2.030 | 2.020 2.006 2.011 2.004 2.010 | 2.010 ± 0.0077 | 2.014 2.023 2.020 2.002 2.008 | 2.013 ± 0.0120 |
| 4-Nitrobenzyl alcohol | 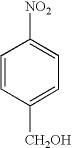 | 1.260 | 1.252 1.264 1.244 1.256 1.268 | 1.257 ± 0.0119 | 1.266 1.295 1.234 1.296 1.250 | 1.268 ± 0.0381 |
| Chlorpromazine | 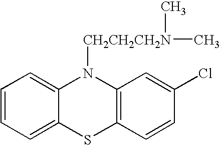 | 3.200 | 3.075 3.074 3.076 3.083 3.074 | 3.076 ± 0.0047 | 3.144 3.179 3.186 3.124 3.186 | 3.164 ± 0.0111 |
| Imipramine | 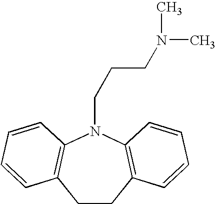 | 2.500 | 2.511 2.579 2.573 2.569 2.571 | 2.561 ± 0.0348 | 2.686 2.445 2.678 2.691 2.446 | 2.589 ± 0.1632 |
| Pyridine | 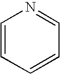 | 0.650 | 0.674 0.699 0.654 0.691 0.689 | 0.681 ± 0.0221 | 0.693 0.687 0.696 0.702 0.704 | 0.696 ± 0.0171 |
| Quinoline | 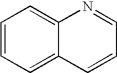 | 2.020 | 2.116 2.115 2.113 2.121 2.114 | 2.116 ± 0.0039 | 2.191 2.185 2.182 2.184 2.188 | 2.186 ± 0.0044 |
| Aniline | 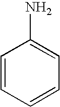 | 0.900 | 0.936 0.945 0.929 0.926 0.932 | 0.934 ± 0.0094 | 0.963 0.990 0.935 0.990 0.989 | 0.973 ± 0.0303 |
| 4-Nitrophenol | 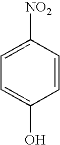 | 1.480 | 1.771 1.779 1.811 1.785 1.780 | 1.785 ± 0.0190 | 1.814 1.803 1.808 1.806 1.805 | 1.807 ± 0.0052 |

### Example 7

### Formation of Nanoparticles with an Enzyme Core

A first buffer solution was prepared comprising potassium dihydrogenphosphate 0.01 mol and sodium chloride 0.25 mol in 500 ml de-ionized water. The buffer pH value was adjusted to 7.0 by addition of sodium hydroxide solution at 20° C.

Penicillinase (β-Lactamase I, purified from Bacillus cereus, Sigma) was then dissolved in a second buffer solution of the same composition to an enzyme concentration of 50 nM. A micro-emulsion was formed as in example 1 (0.02 mol CTAB in 100 g dried toluene) to which 5.2 g of the first buffer solution was added slowly in droplets with continuous stirring. 2 ml of the second buffer solution (containing penicillinase) was added over 4 hours. The mixture was then stirred for a further four hours to ensure an even dispersion of enzyme molecules in the micro-emulsion system. A 200 μl sample of the mixture was removed for comparison of enzymatic activity (see example 8). 6.94 g TEOS was then slowly added to the system, which underwent hydrolysis at the water/toluene interface to form the external silica coating.

### Example 8

### Enzymatic Activity Test on the Product of Example 7

The 200 μl sample of the mixture removed prior to addition of TEOS in example 7 was analysed, using UV-visible spectroscopy to follow the hydrolysis of the lactam group of pencilling at 232 nm, to determine whether the enzyme is still functional through hydrolysis of a calibrated standard penicillin V (Phenoxymthylpenicillinic acid) (3 nM, Sigma). A further 200 μl sample of the mixture from example 7 was extracted six days after addition of the TEOS and analysed in the same manner.

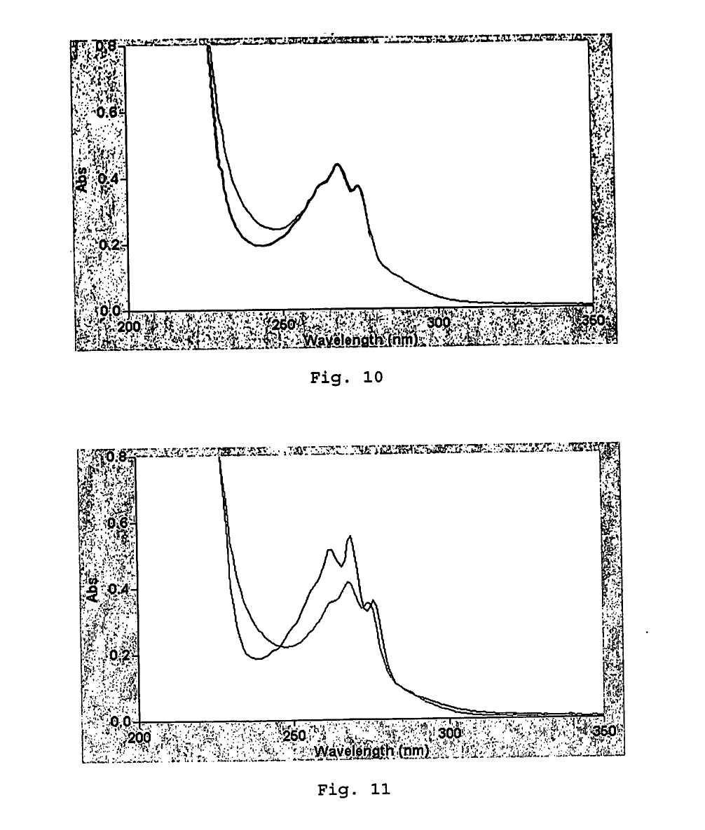

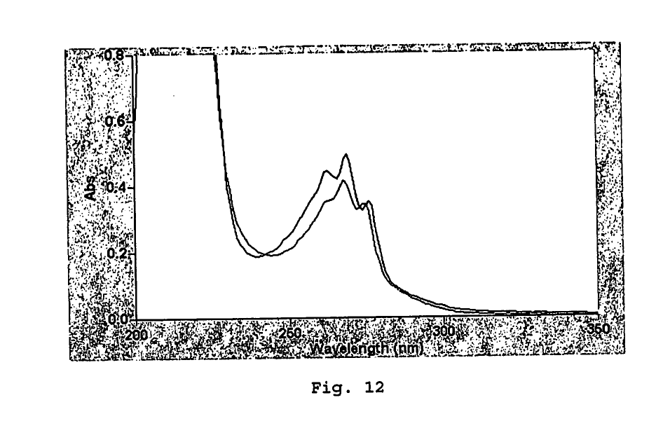

UV-visible spectra are shown in FIGS. 10 to 12. The spectral curves represent principally the UV-visible spectra of the penicillin V. Hydrolysis of Penicillin V by the added free form of β-Lactamase I showing a typical UV-visible spectral change is-plotted in FIG. 10. It can be clearly seen that the absorbance value at 232 nm (the region where lactam group absorbs) decreases over five minutes indicating that rapid conversion of the penicillin V to corresponding penicilloic acid is occurring (hydrolysis of the lactam group). FIG. 11 shows the result of penicillin V solution upon the addition of the 200 μl sample extracted from the reaction mixture before TEOS addition (no silica coat). The absorbance value at 232 nm where the lactam group is located is again attenuated over the 5 minute period shown. This indicates that the enzyme remains active when incorporated into micelles of the mixture. FIG. 12 also shows that a similar spectral change is observed when using the sample extracted six days after TEOS addition to the mixture of example 7 (silica coated nanoparticles). The absorbance value at 232 nm clearly continued to decrease over the five minutes between the two spectra.

These results indicate that the entrapped enzyme remains functional in both the micelle and the silica-coated composite environments.

Next there will be described details of the method of the invention of depositing a component into a porous material, in particular nanoparticles, using a supercritical fluid.

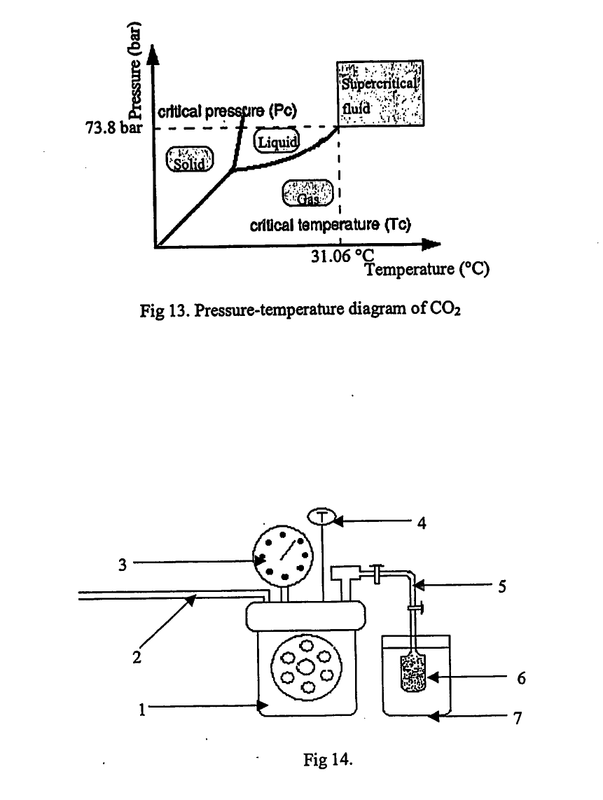

The supercritical conditions for any compound can be achieved at a temperature and pressure which are at or above its critical values. The critical temperature of a compound is defined as the temperature above which a pure, gaseous component cannot be liquefied regardless of the pressure applied. The critical pressure is then defined as the vapour pressure of the gas at the critical temperature. The temperature and pressure at which the gas and liquid phases become identical is the critical point. In the supercritical environment only one phase exists. The fluid, as it is termed, is neither a gas nor a liquid and is best described as intermediate between these two extremes. This phase retains the solvent power common to liquids as well as the transport properties common to gases. Carbon dioxide is the most commonly known supercritical fluid. The pressure-temperature diagram for carbon dioxide is presented in FIG. 13 to illustrate the differences between the gas, liquid and supercritical states. As seen from FIG. 13, pure CO₂ can be liquefied by compression only at temperatures below 31.060C. Above this critical temperature TC and the corresponding critical pressure PC (73.8 bar), no distinct liquid or gaseous phases exists. This is referred as to supercritical region, where SC—CO₂ exists.

The properties of SC—CO₂ depend on the temperature and pressure but are generally intermediate to those of gas and liquid state. Supercritical CO₂ has its own advantages as an environmentally friendly solvent. The ability to dissolve chemical compounds is one of its main properties. The solubility of chemical compounds in SC—CO₂ is affected by temperature and pressure. The solubility of n-octanol in SC—CO₂ was investigated in 2001 (H. Nakaya, O. Miyawaki and K. Nakamura, Enz. Micro. Tech., 28, 176-182, 2001). In this work, the log P of supercritical CO₂ in n-octanol/water system was determined from the solubility of n-octanol in CO₂. The solubility of SC—CO₂ in n-octanol is about 1.0 M at 55 bar, 50° C. The solubility is increased while the pressure is increased. This indicates that the n-octanol has a good solubility in SC—CO₂. SC—CO₂ shows a substantial higher diffusivity and lower viscosity than liquid CO₂. Because of the density, its viscosity and diffusivity are dependent on temperature and pressure. The diffusivity and viscosity curves of the SC—CO₂ at different pressure and temperature are shown in M. A. McHugh and V. J. Krukonis, Supercritical Fluid Extraction-Principles and Practice, Butterworths, Boston, Mass., 10, 1986 and S. V. Kamat, B. Iwaskewyct, E. J. Berkman and A. J. Russell, Proc. Natl. Acad. Sci., 90, 2940, 1993. It is also shown that diffusivity increases with an increase in temperature or a decrease in pressure. In contrast to diffusivity, the viscosity is shown to decrease with an increase in temperature or a decrease in pressure.

The advantages of SC—CO₂, of high n-octanol solubility, high diffusivity and low viscosity are employed in the following example for the delivery of n-octanol to porous nanocomposites via the supercritical medium. The result is a homogeneous solution containing the magnetic nanocomposites with evenly charged octanol.

### Example 9

### Charging n-Octanol to Porous Nanocomposites Via SC—CO₂ Delivery and Preparation of Stock Solution

In the following examples, n-octanol (99%), potassium diydrogenphosphate, 4-nitroanisole (97%), 4-nitrobenzyl alcohol (99%) and 4-nitrophenol (98%) were obtained from Aldrich. Imipramine, quinoline, chlorpromazine, benzamide were obtained from Sigma in analytical grade quality or above. All of these chemicals were used without further purification.

The charging n-octanol to the porous nanocomposites was carried out using the set-up shown in FIG. 14.

FIG. 14 shows an autoclave 1 to which high-pressure CO₂ is delivered via a pipe 2. The autoclave 1 has a pressure detector 3 and a temperature controller 4 to maintain constant temperature. The autoclave 1 is connected by a valved conduit 5 to a sample holder 6 which is held in a water bath 7.

0.0607g of the dried porous silica encapsulated nano-composites prepared as in Example 1 above was placed into the sample holder 6. 30 μL n-octanol was then added into the holder 6 by the use of a micro-pipettor. The autoclave vessel and the sample holder (30 ml in total volume) were charged and flushed with CO₂ by opening and closing the valves between the two compartments and external outlets for a few times before the vessels were brought up to the desired pressure (150 bar). The temperature of the autoclave and sample holder was maintained at 40° C., to allow the dispersion of the small quantity of n-octanol into the porous particles. After 2 hours, the high pressure of the system was released to atmosphere very slowly. By measuring the weight change of the sample holder, it was found that 0.0155 g of n-octanol (density 0.8240 g/mL) was adsorbed in the particles, which amounts to 18.8 μL.

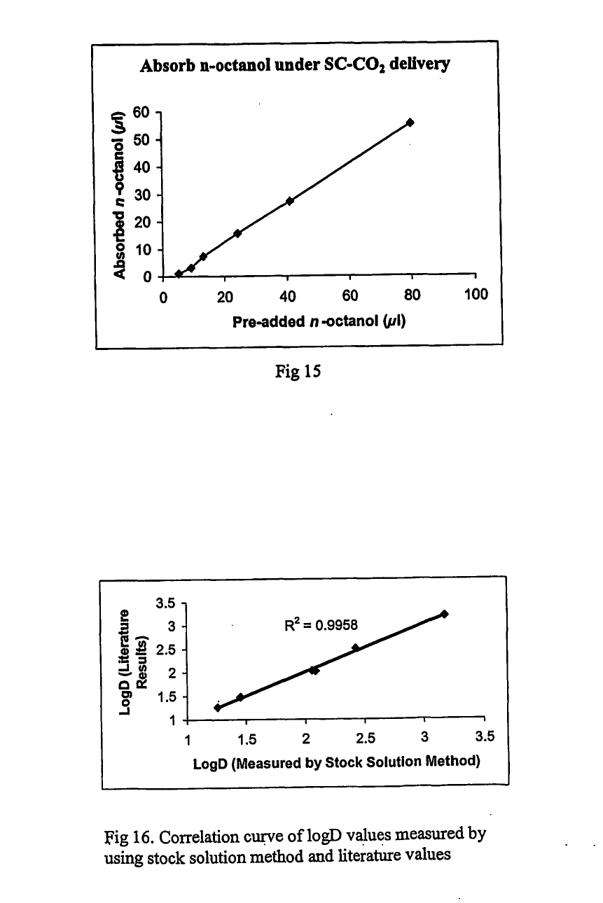

Owing to the relatively high solubility of n-octanol in SC—CO₂, some of the n-octanol was lost during the depressurization process. The composite powder carrying the n-octanol appeared to be light and dry in contrast with those samples prepared through the direct mixing of the solvent to the dried powder. The plot in FIG. 15 shows the relationship between the absorbed amount of n-octanol measured (weight gained) and the amount of n-octanol added to the same amount of particles, in several similar experiments.

In an alternative procedure, the n-octanol is placed in the autoclave 1 and dissolves in the SC—CO₂ in the autoclave before the SC—CO₂ is brought into contact with the particles in the sample holder 6. Both procedures appear to produce similar results.

Without wishing to be bound by theory, we can suppose that the SC-fluid, with gas-like character, penetrates very freely into the pores of the particles (compared with a liquid) and that on depressurisation the decrease of solubility of the dissolved component allows it to condense on the large surface area of the pores of the particles (which is much larger than the exterior area of the particles or the surface area of the reaction vessel).

To prepare a stock solution of these particles carrying n-octanol in an aqueous solvent medium, a buffer solution containing 0.1 mM potassium diydrogenphosphate, pH=7.4 was initially prepared and placed in a separatory funnel. An appropriate amount of n-octanol was added to provide a thin n-octanol layer covering the water phase (density of n-octanol is lower than water). The funnel was shaken for 5 to 10 minutes to allow mixing of the n-octanol with water. The funnel was then covered by aluminium foil to protect the solvent mixture from light degradation and evaporation. The funnel was placed in an upright position for 3 days to allow separation of the two phases. The n-octanol phase saturated with water was then collected.

The particles containing nanocomposite were dispersed into 5 mL of this saturated buffer solution to obtain a homogeneous stock-solution with a concentration of 0.0038 mL n-octanol per mL of solution. Partition coefficient measurements were carried out as described below, using such a stock solution prepared in the same way.

### Example 10

### Partition Coefficient Determination Using Stock Solution

In each experiment, 1 mL of the stock solution of n-octanol containing nanoparticles, prepared by the method using SC—CO₂, was extracted by micro-pipettor and mixed with 3 mL analyte solution. After shaking the mixed system for half an hour, the magnetic nanocomposites were precipitated in a few minutes by an external magnet placed near the bottom of the reactor-tube. UV-visible spectrophotometry was used to determine the absorption of analyte at the aqueous phase both before and after partitioning. Partition coefficients (log D) of seven analytes were measured by this method and are given in table 3 below together the log D values of these analytes at pH=7.4 determined by the traditional shake flask method and the results obtained in example 6 above.

For the partition coefficient determination, stock solutions were prepared with a suitable n-octanol/nanoparticle ratio. A typical ratio of 0.071 g nanocomposite to 40 μl n-octanol gave, after the SC—CO₂ delivery, actually 22.3 μl n-octanol on the nanocomposites. The stock solution was prepared by directly dispersing these n-octanol pre-absorbed nanocomposites into 5 ml potassium phosphate buffer solution (pH=7.4). In the work here described, a typical volume concentration of n-octanol in buffer solutions is 0.0045 ml n-octanol per ml of buffer solution.

UV-visible spectrometry was used to quantitatively determine the drug concentration in aqueous phase before and after partitioning. The n-octanol/water partition coefficient (log D) is defined as the ratio of the activities of a species in the two phases at equilibrium. At a great dilution we use the concentration to replace the activity. This definition can be described as the equation below:
log D=log [Co/Cw]
where Co and Cw are the drug concentration in n-octanol and aqueous phase after establishing the partitioning equilibrium.

As mentioned, for log D measurement, 1 mL of stock solution was mixed with 3 mL of initial drug solution. Since the concentration of n-octanol in buffer solution is 0.0045 mL n-octanol per mL of the stock solution, the volume ratio of water/n-octanol in the measurement is about 889:1. This ratio is much higher than the normal ratio of 100:1 used in the shake flask method, which enables reliable determination of high log D values. It is difficult to separate such a small n-octanol phase by the shake flask method but it seems to be greatly facilitated by using the magnetic nanocomposites.

Table 3 lists the results of the log D values measured independently by using the stock solution method of the present examples, the shake flask method and the magnetic nanocomposite method of example 6. Each drug analyte was measured at least by five times by all three methods and the average measured log D value is shown. The water/n-octanol volume ratio as 100 was used in the shake flask method as well as the magnetic nanocomposite method. The statistical confidence level is at 95%. The log D value of each drug analyte obtained from literature is also listed for comparison.

TABLE 3
Comparison of n-octanol-water partition coefficients (logD)
(Confidence level is 95%)

| | | Stock | Magnetic nano- | | |
|---|---|---|---|---|---|
| | | solution | composite | Shake-Flask | Literature |
| Drug Analyte | Structure | method | method | Method | Value* |
| 4-Nitroanisole | 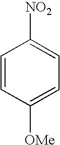 | 2.054 ± 0.068 | 2.013 ± 0.012 | 2.010 ± 0.008 | 2.030 |
| 4-Nitrobenzylalcohol | 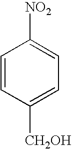 | 1.263 ± 0.010 | 1.268 ± 0.038 | 1.257 ± 0.012 | 1.260 |
| Chlorpromazine | 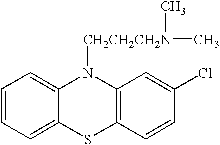 | 3.175 ± 0.448 | 3.164 ± 0.011 | 3.076 ± 0.005 | 3.200 |
| Imipramine | 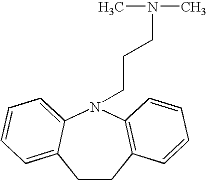 | 2.426 ± 0.490 | 2.589 ± 0.163 | 2.561 ± 0.035 | 2.500 |
| 4-Nitrophenol | 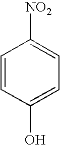 | 1.453 ± 0.026 | 1.491 ± 0.021 | 1.486 ± 0.009 | 1.480 |
| Quinoline | 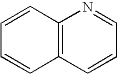 | 2.082 ± 0.073 | 2.186 ± 0.004 | 2.116 ± 0.004 | 2.020 |
| Benzamide | 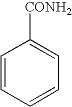 | 0.703 ± 0.127 | 0.678 ± 0.046 | 0.654 ± 0.009 | 0.660 |

*The literature log D values of the drug compounds at pH = 7.4 were from AstraZeneca in-house data

From the results above, the log D values of the drug molecules within the range from 1.260 to 3.20 were successfully measured by all three methods. As seen from the data, most log D values obtained from the stock solution method correlate well with the literature values. Since a high water/n-octanol volume ratio was used in our measurement, the limitation in sensitivity particularly for those drug molecules with low log D values is observed. The drug compound with low log D value, which shows a low solubility in n-octanol phase would not give much change in their concentration in aqueous phase even after partitioning. As a result, the accuracy of the experimental results for low log D compounds will highly depend on the phase ratios used (relate to the quantity of stock solution added) and the sensitivity of the detection technique involved. A slight error in concentration measurements will cause a significant change in the final log D values obtained. A typical example is the benzamide whose log D value is 0.66 at pH=7.4. It is expected a smaller degree of error will be achieved by either enhancing the sensitivity of the analysis method or by reducing the volume ratio of water/n-octanol used.

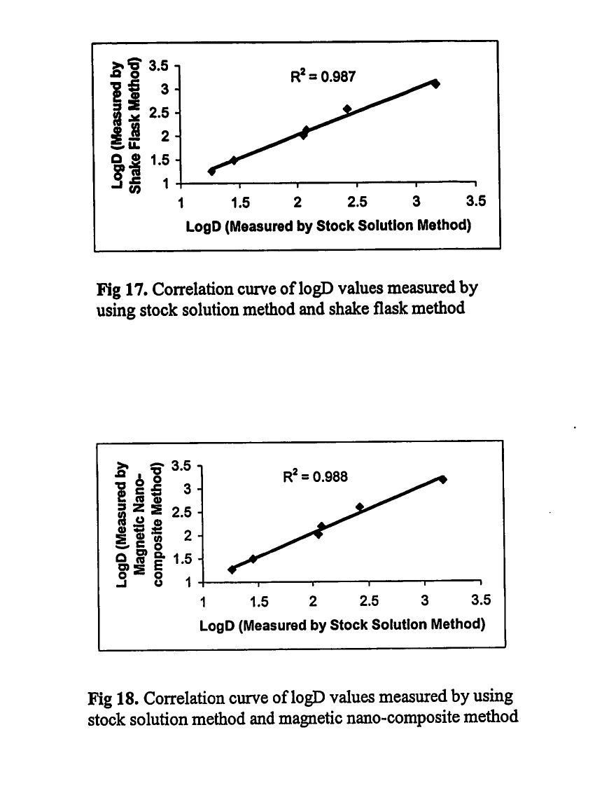

A correlation curve of the results from this present method and the accepted values from literature is presented in FIG. 16 (exclude the benzamide data). The correlation coefficient of this curve is found to be 0.9958. FIG. 17 shows the correlation curve of the results from the stock solution method of this example with those from the standard shake-flask method. The correlation coefficient of this curve is 0.987. FIG. 8 shows the correlation curve of the results from this stock solution method of this example and the magnetic nanocomposite method of example 6. The correlation coefficient of this curve is 0.988. All these indicate the reliability of the stock solution method, which clearly suggest that the homogeneous dispersion/deposition of n-octanol onto porous nanocomposites can be successfully achieved using supercritical carbon dioxide. In table 3, the statistic deviations of the log D values measured by the stock solution method are slightly larger than the other two methods at the same confidence level.

In a procedure where the log D value of the compound being tested is not known, it may be preferable to perform a parallel test on a compound or compounds of known log D using the same stock solution containing nanoparticles, in order to check the amount of the second solvent (e.g. n-octanol) contained in the stock solution by back calculation.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

1. A method of measuring the partition coefficient of a compound between two immiscible solvents, said method comprising the steps of:
a) providing a composition which contains said compound and comprises nanoparticles having a porous surface and a first solvent, wherein a second solvent is absorbed into the pores of the nanoparticles and wherein said first and second solvents are immiscible;
b) separating the product of step a) into two components, the first comprising the nanoparticles and the second comprising the solvent; and
c) determining the partition coefficient from the partition of the compound between said first and second components.

2. A composition comprising nanoparticles having a porous surface and a first solvent, wherein a second solvent is absorbed into the pores of the nanoparticles and wherein said first and second solvents are immiscible.

3. A composition according to claim 2 wherein said nanoparticles form a colloidally stable suspension in said first solvent.

4. A composition according to claim 2 or 3 wherein said porous surface is formed of any one of silica, alumina, titania, zirconia or carbon.

5. A composition according to any one of claims 2 to 4 claim 2 wherein the nanoparticles further comprise a magnetic material core.

6. A composition according to claim 5 wherein said magnetic material core is formed from magnetite (Fe₃O₄), maghemite (γFe₃O₄), greigite (Fe₃S₄), Fe₂CoO₄, a ferromagnetic metal or alloy or carbide.

7. A composition according to claim 2 wherein said nanoparticles have a diameter of between 2 nm and 1 μm.

8. A composition according to claim 2 wherein the porous surface layer of said nanoparticles has a thickness of between 1 nm and 100 nm.

9. A composition according to claim 2 wherein said first solvent is aqueous, particularly is water.

10. A composition according to claim 2 wherein said second solvent is one of n-octanol, cyclohexane, a C₆-C₁₀ alkane, chloroform, propylene glycol dipelargonate (PGDP), 1,2-dichloroethane, olive oil, benzene, toluene, nitrobenzene, chlorobenzene, tetrachloromethane, oleyl alcohol, 4-methylpentan-2-ol, pentan-1-ol, pentan-2-ol, isobutanol, butan-1-ol, 2-methylbutan-2-ol, butan-2-ol, butan-2-one, diethyl ether, isoamyl acetate, ethyl acetate, etc. or a monophasic mixture of two or more of these.

11. A composition according to claim 2 wherein the volume ratio of said first solvent to said second solvent is between 3000:1 and 1:1 (preferably in the range 500:1 to 50:1).

12. A composition according to claim 11 wherein the ratio of said first solvent to said second solvent is at least 100:1.

13. A method of attaining partition of a compound between two immiscible solvents comprising incorporating said compound in a composition according to claim 2.

14. A composition for use in a quantitative analytical technique, comprising nanoparticles each having a porous surface and a solvent adsorbed in the pores of the nanoparticles in a predetermined amount per unit weight of the composition.

15. A composition according to claim 14, wherein said porous surface is formed from any one of silica, alumina, titania, zirconia or carbon.

16. A composition according to claim 14 or 15 wherein the nanoparticles each have a magnetic material core.

17. A composition according to claim 14, wherein said solvent is immiscible with water.

18. A composition according to claim 17 wherein said second solvent is one of n-octanol, cyclohexane, a C₆-C₁₀ alkane, chloroform, propylene glycol dipelargonate (PGDP), 1,2-dichloroethane, olive oil, benzene, toluene, nitrobenzene, chlorobenzene, tetrachloromethane, oleyl alcohol, 4-methylpentan-2-ol, pentan-1-ol, pentan-2-ol, isobutanol, butan-1-ol, 2-methylbutan-2-ol, butan-2-ol, butan-2-one, diethyl ether, isoamyl acetate, ethyl acetate, etc. or a monophasic mixture of two or more of these.

19. (canceled)

20. A method of measuring the partition coefficient of a compound between two immiscible solvents, said method comprising the steps of:
a) incorporating said compound in a composition according to claim 2;
b) separating the product of step a) into two components, the first comprising the nanoparticles and the second comprising the first solvent; and
c) determining the partition coefficient from the partition of the compound between said first and second components.

21. A method according to claim 1 or 20 wherein step c) comprises determining the amount of said compound which remains in said first solvent.

22. A method according to claim 1, or claim 20 wherein said compound is a bioactive drug molecule.

23. A method according to claim 1 or claim 20 wherein step b) is performed by any one of filtration, centrifugation and magnetic separation.

24. A method according to claim 1 or claim 20 wherein step c) comprises recording the UV-visible spectrum of said supernatant solution.

25. A method according to claim 1 or claim 20 further comprising shaking the composition of step a) prior to performing the separation step b).

26. A nanoparticle having a core comprising a catalytically active species, and a porous layer surrounding the core which has a pore size such that the catalytically active species is entrapped.

27. A nanoparticle according to claim 26 wherein said core catalytically active species is a biologically active species, e.g. an enzyme or other protein.

28. A nanoparticle according to claim 27 wherein said biologically active species is any one of blood serum albumin, β-Lactamase I (Penicillinase),kinase, a carboxylesterase, metallothionin, cytochrome b, c, P450, etc.

29. A nanoparticle according to claim 26 wherein said porous layer is formed from any one of silica, alumina, titania, zirconia or carbon.

30. A nanoparticle according to claim 26 wherein said core further comprises a magnetic material.

31. A nanoparticle according to claim 30 wherein said magnetic core is formed from magnetite (Fe₃O₄), maghemite (γFe₃O₄), greigite (Fe₃S₄) or Fe₂CoO₄ or ferromagnetic metal or alloys (such as Fe—Pt, Fe—Co, Fe—Ni), metal carbides, etc.

32. A nanoparticle according to claim 26 wherein said nanoparticles have an average a diameter of between 2 nm and 1 μm.

33. A nanoparticle according to claim 26 wherein the core of the nanoparticle has an average diameter of between 1 and 10 nm.

34. A nanoparticle according to claim 26 wherein the porous outer coating on said nanoparticle has a thickness between 1 nm and 100 nm.

35. An assembly of nanoparticles at least some of which are nanoparticles according to claim 26, wherein on average the number of molecules of said catalytically active species per nanoparticle of the assembly is not more than one.

36. A method of making a nanoparticle according to claim 26, comprising the following steps:
a) forming, in a liquid medium, colloidal particles containing the catalytically active species to be contained in the nanoparticle core, the particles being colloidally stabilised by a surfactant;
b) treating said colloidal particles by hydrolysis or pyrolysis to form the porous layer surrounding the catalytically active species.

37. A method of claim 36 wherein, in step a), said colloidal particles further contain a magnetic material or a precursor to a magnetic material.

38. A method of claim 36 or 37 wherein said colloidal particles comprise aqueous colloidal particles in a solvent which is immiscible with water.

39. A method of claim 38 further comprising adding a salt of silicon, aluminium, titanium or zirconium to the product of step a), which forms the corresponding oxide compound upon hydrolysis at the colloid boundary.

40. A method of claim 39 wherein said silicon salt is tetraethyl orthosilicate (TEOS) and the surfactant is cetyltrimethylammonium bromide (CTAB).

41. A method of depositing a component in pores of a porous material, by contacting the porous material with a solution of the component in a supercritical fluid.

42. A method according to claim 41 wherein the supercritical fluid is removed by depressurising it and allowing it to evaporate.

43. A method according to claim 1 or 2 wherein the component is a liquid.

44. A method according to claim 41 wherein the component is substantially insoluble in water.

45. A method according to claim 41 wherein the porous material is porous particles.

46. A method according to claim 45 wherein the porous particles are nanoparticles, having a particle size not greater than 1 μm.

47. A method according to claim 41 wherein the porous material has a porous silica surface.

48. A method according to claim 41 wherein the supercritical fluid is carbon dioxide.

49. A method of preparing a composition containing two components comprising preparing porous particles containing a first component in a predetermined amount by a method according to claim 45, and adding said particles to a liquid second component.

50. A method according to claim 49 wherein the first and second components are immiscible.
